(19) United States
(12) Patent Application Publication
(10) Pub. No.: US 2009/0118583 A1
(43) Pub. Date: May 7, 2009

(54) SWITCHING MECHANISM FOR ALTERING OPERATION WITH LIGHT

(75) Inventor: Kentaro MATSUMOTO, Saitama (JP)

Correspondence Address: GREENBLUM & BERNSTEIN, P.L.C., 1950 ROLAND CLARKE PLACE, RESTON, VA, 20191, US

(73) Assignee: HOYA CORPORATION, Tokyo (JP)

(21) Appl. No.: 12/264,357

(22) Filed: Nov. 4, 2008

(30) Foreign Application Priority Data
Nov. 7, 2007 (JP) 2007-289898

(51) Int. Cl. A61B 1/00; G11C 13/04

(52) U.S. Cl. 600/118; 365/106

(57) ABSTRACT

A switching mechanism is used for altering the operation of an electronic device. The switching mechanism has a memory comprising a plurality of memory cells. Each memory cell undergoes a change in binary data upon receiving more than a specific amount of light having a specific wavelength. The operation is altered when the binary data of at least one memory cell of the plurality of memory cells is changed by the light.

### BACKGROUND OF THE INVENTION

1. Field of the Invention

The present invention relates to a switching mechanism for a swallowable medical device such as a capsule endoscope, for example, and in particular, to a mechanism which can alter the operation of a medical device via a UV-EPROM.

2. Description of the Related Art

A capsule endoscope is well known, as an endoscope for observing the insides of a human body such as the stomach or intestines. Circuits in the capsule endoscope are driven by a power-supply unit such as a battery which is provided, in the capsule endoscope, but they can be driven only for a short period by the battery because of the limited available charge. Therefore, the power supply of the capsule endoscope is usually turned on by the doctor just before being swallowed by the patient, in order to reduce unnecessary power consumption. Furthermore, it is preferable that the endoscope be turned on by a simple operation and without disassembling in order to ensure the seal of the interior.

Conventionally, a switching mechanism for a capsule endoscope utilizing a photo-interrupter is known as a means for turning on the power supply of the capsule endoscope, as shown in Japanese Unexamined Patent Publication (KOKAI) No. 2005-278815. In this mechanism, the photo-interrupter is provided, in the endoscope and faces a dome-shaped transparent cover of the capsule endoscope. When the photo-interrupter receives light passing through the transparent cover, the capsule endoscope is turned on. While this endoscope is stored, the transparent cover is covered by a protective cap in order to prevent light from entering the photo-interrupter. When it is used, the protective cap is released in order to illuminate the photo-interrupter.

Furthermore, as shown in Japanese Unexamined Patent Publication (KOKAI) No. 2005-73934, there is known another switching method utilizing a light sensor. In this mechanism, when a light sensor which is provided in the endoscope receives light having a predetermined emission pattern, the capsule endoscope is turned on or off.

However, the above-mentioned switching mechanisms require a special member such as a photo-interrupter and a light sensor which is used exclusively for turning the capsule endoscope on or off, which complicates the structure of the endoscope.

### SUMMARY OF THE INVENTION

Therefore, an object of the present invention is to provide a switching mechanism, which can alter the operation of a device without the special member used exclusively for altering the operation.

According to the present invention, there is provided a switching mechanism for switching an operation of an electronic device. The switching mechanism has a memory comprising a plurality of memory cells. Each memory cell undergoes a change in binary data upon receiving more than a specific amount of light of a specific wavelength. The operation is altered when the binary data of at least one memory cell of the plurality of memory cells is changed by the light.

### BRIEF DESCRIPTION OP THE DRAWINGS

The objects and advantages of the present invention will be better understood from the following description, with reference to the accompanying drawings in which:

FIG. 1 is a schematic view of a capsule endoscope in the first embodiment of the present invention;

FIG. 2 is a side view partly showing the capsule endoscope in the first embodiment;

FIG. 3 is a circuit diagram partly showing the capsule endoscope in the first embodiment;

FIG. 4 is a circuit diagram partly showing the capsule endoscope in the second embodiment;

FIG. 5 is a circuit diagram partly showing the capsule endoscope in the third embodiment;

FIG. 6 is a circuit diagram partly showing the capsule endoscope in the fourth embodiment; and

FIG. 7 is a flowchart showing a routine for detecting the amount of ultraviolet exposure.

### DESCRIPTION OF THE PREFERRED EMBODIMENTS

The present invention will be described below with reference to the embodiments shown in the drawings.

A switching mechanism for the capsule endoscope is explained below, but the switching mechanism in these embodiments can be applied to any other swallowable medical devices or any other devices.

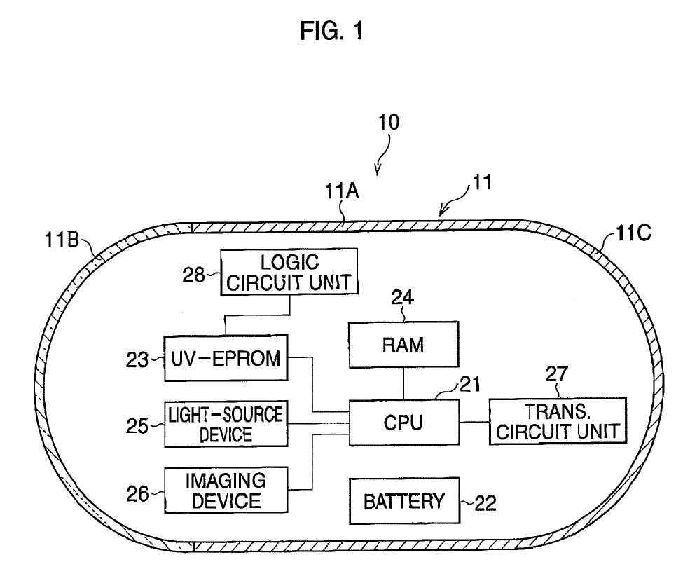

FIG. 1 shows a capsule endoscope having a switching mechanism of a first embodiment. The capsule endoscope 10 is a swallowable medical device for observing the inside of a human body and which enters the human body by being swallowed. The capsule endoscope 10 has a shell 11 which seals its interior. The shell 11 has a body 11A which is opaque and cylindrical, a transparent cover 11B which covers one end of the body 11A, and an opaque cover 11C which covers the other end of the body 11A. The covers 11B and 11C are dome-shaped.

The capsule endoscope 10 has a CPU 21, a UV-EPROM 23, a RAM 24, a light-source device 25, an imaging device 26 such as a CCD or CMOS, a transmitting circuit unit 27, a logic circuit unit 28, and a battery 22 which provides the electrical power for these devices, in the shell 11.

The UV-EPROM 23 is a nonvolatile memory, and data stored therein is erased by ultraviolet light (namely, light of a specific wavelength). The UV-EPROM 23 stores a program for controlling the light-source device 25, the imaging device 26, the transmitting circuit unit 27, and so on. The CPU 21 is driven while the electrical power is supplied thereto. The driven CPU 21 operates and controls the operations of several devices including the light-source device 25, the imaging device 26, the transmitting circuit unit 27, and so on, based on the program recorded in the UV-EPROM 23.

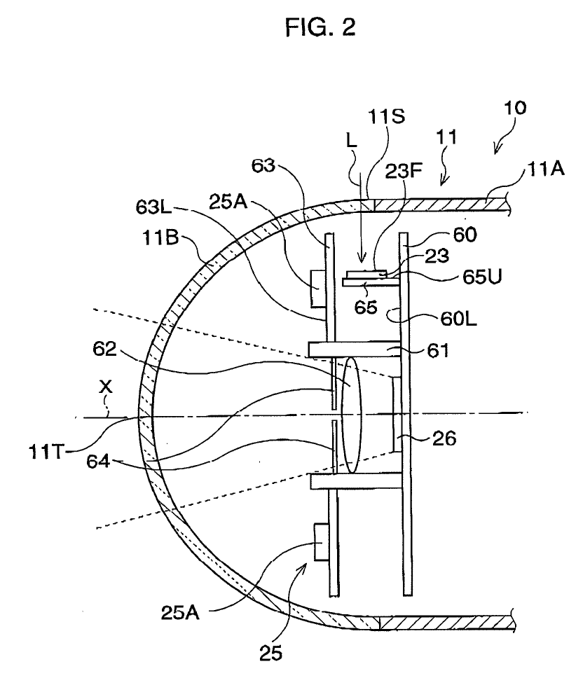

Following the program, the light-source device 25 illuminates an object with light which passes through the transparent cover HE to the exterior of the endoscope 10. The imaging device 26 captures an object image which is formed thereon by an object lens system 62 (refer to FIG. 2) and generates an image signal from the object image. The transmitting circuit unit 91 transmits the image signal to the exterior of the capsule endoscope 10 by radio waves. The RAM 24 temporarily stores the necessary data for the operations of these devices.

FIG. 2 is a side view partly showing the capsule endoscope 10 in the first embodiment. As shown in FIG. 2, a circuit substrate 60 is provided in the shell 11, and the imaging device 26 is mounted on a front surface 60L of the circuit substrate en. The circuit substrate 60 is perpendicular to the main axis X of the shell 11 (namely, the body 11A) extending in the longitudinal direction of the shell 11, and the imaging device 26 is arranged on the main axis x. A lens barrel 61 is also mounted on the front surface 60L and holds the objective lens system 62 therein such that the objective lens system 62 is arranged in front of the imaging device 26 on the main axis X. The optical axis of the objective lens system 62 is identical with the main axis X. The objective lens system 62 is oriented toward a top portion 11T of the transparent cover 11B. The top portion 11T is on the main axis. The lens system 62 receives light passing through the transparent cover 11B from the object so as to form the object image on the imaging device 26.

The lens barrel 61 also holds a diaphragm 64 therein. The diaphragm 64 is arranged in front of the lens system 62 along the optical axis, and can adjust the amount or light incident onto the lens system 62. A light source substrate 63 is disposed in front of the circuit substrate 60. The light source substrate 63 surrounds the lens barrel 62 and is fixed on the outer surface of the lens barrel 62. The light-source device 25 includes two or more light-emitting elements 25A. The light-emitting elements 25A are mounted on a front surface 63L of the substrate 63 such that light-emitting elements 25A are disposed around the lens barrel 62.

A memory substrate 65 which is mounted on the front surface 60L is arranged at a position which does not overlap the main axis X. The memory substrate 65 is parallel to the main axis X (the optical axis of the lens system 62) and the UV-EPROM 23 is mounted on a surface 65U of the substrate 65. The surface 65U orients outwardly in a radial direction of the body 11A. Due to this, the UV-EPROM 23 is arranged parallel to the main axis X, and a surface 23F of the UV-EPROM 23 orients outwardly in the radial direction, namely, orients in a direction other than the direction in which the objective lens system 62 is oriented. The surface 23F is oriented towards an edge portion 11B of the transparent cover 11B which connects to the body 11A.

The surface 23F (namely, memory cells 33 provided thereon, as described below) can receive light passing through the transparent cover 11B from the exterior of the shell 11, and in particular, can receive light traveling perpendicularly to the main axis X most efficiently. On the other hand, the objective lens system 62 does not receive that light because the optical axis of the lens system 62 is parallel to the main axis X. Accordingly, ultraviolet light L, which is emitted parallel to or at a small tilt with respect to a perpendicular direction of the main axis X from the exterior of the shell 11 to the shell 11 by a UV-emitting device (not shown in the Figures) is received by the UV-EPROM 23, but not by the objective lens system 62 (namely, the imaging device 26). In other words, the objective lens system 62 can be isolated from the ultraviolet light L directed from outside the shell 11 to the UV-EPROM 23.

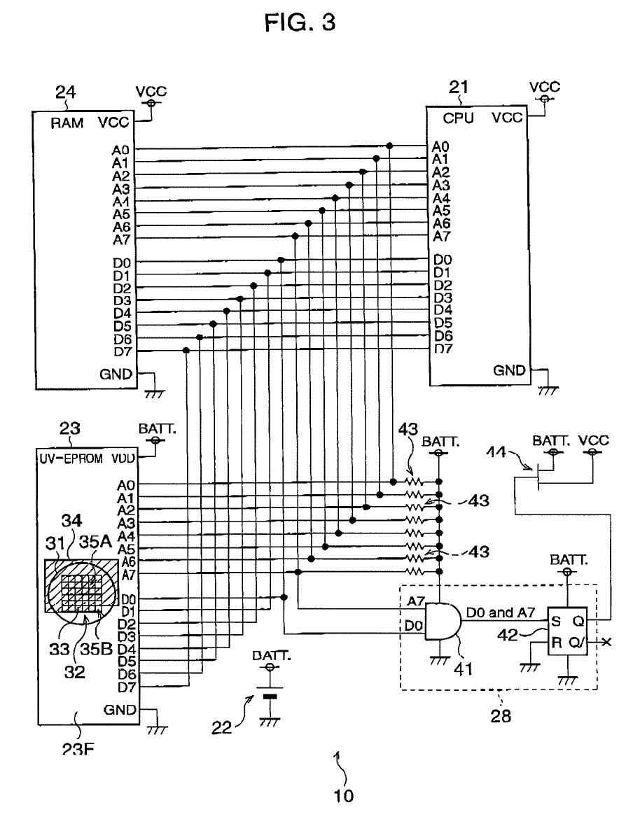

FIG. 3 is a circuit diagram partly showing the capsule endoscope 10 in the first embodiment. In FIG. 3, for simplicity, address decoders are not shown. As shown in FIG. 3, the CPU 21 connects to the RAM 24 and the UV-EPROM 23 through address lines A0-A7 and data lines D0-D7. The CPU 21 transmits data from the RAM 24 and the UV-EPROM 23 to the CPU 21 or from the CPU 21 to these through address lines A0-A7 and data lines D0-D7.

The UV-EPROM 23 has a transparent window 31 and a bare chip 32. The transparent window 31 which is composted of a transparent material such as glass, can transmit at least ultraviolet light. The transparent window 31 is provided at the surface 23F. The bare chip 32 is arranged beneath the transparent window 31, and a plurality of memory cells 33 are provided on the bare chip 32.

As described above, the plurality of memory cells 33 can receive light from the exterior of the shell 11 through the transparent cover 11B and the windows 31. However, a part of the transparent window 31A is covered by a shield member 34 which is made of an opaque material in this embodiment. Therefore, some memory cells (shielded memory cells) of the plurality of memory cells 33 are shielded by the shield member 34 such that the shielded memory cells cannot receive light from the exterior of the shell 11. The shielded memory cells form a shielded memory field 35A. The exposable memory cells are not shielded by the shield member 34 and are exposable by light from the exterior of the shell 11 through the transparent cover 11B and the window 31. The exposable memory cells form an exposable cell field 35B.

Each memory cell 33 has a floating gate which can store electrical charge. The binary value of the memory cell 33 is zero when the amount of stored charge in its floating gate exceeds a specific amount, and it is one when it does not. The memory cell 33 releases the stored charged when it receives ultraviolet light, and the binary data of the memory cell 33 changes from one to zero. Namely, each memory cell 33 can undergo a change in binary data upon receiving more than a specific amount of ultraviolet light. The UV-EPROM 23 can therefore store specific data using binary data in the memory cells 33. The capacity of each UV-EPROM 23 and RAM 24 is 256 bytes.

The UV-EPROM 23 stores specific data constituting the above-mentioned program (program data) in the shielded memory field 35A. Because the shielded memory field 35A (the shielded memory cells) cannot receive light from the exterior of the shell 11, the binary data in the shielded memory field 35A does not change. Accordingly, the program data is not deleted from the shielded memory field 35A by the light from the exterior of the shell 11. In this embodiment, the whole memory field except for the final byte (1-byte memory field) whose address is 0ffh constitutes the shielded memory field 35A. Namely, the 255-byte memory field with addressee 00h-Ofeh is allocated to the shielded memory field 35A.

The final byte (address 0ffh) which is not shielded by the shield member 34 is allocated to the exposable memory field 35B. The exposable memory field 35B is used for altering the operation of the capsule endoscope 10 as described below, and not for storing data such as program data.

The logic circuit unit 28 has an AND circuit 41 and an SRFF (set/reset flip-flop) 42. The AND circuit 41 has two input terminals which connect to the address line A7 and the data line D0, respectively. The AND circuit 41 outputs a high-level signal to the SRFF 42 through an output terminal when high-level signals are input to both the input terminals.

The SRFF 42 has a set terminal S which connects to the output terminal of the AND circuit 41, a reset terminal R which is grounded, and a non-inverted output terminal Q which is connected to a FET 44. The SRFF 42 outputs a high-level signal through the output terminal Q, when the high-level signal is input to the SRFF 42 from the AND circuit 41 through the set terminal S. However, the SRFF 42 does not flip the level of the signal which is output through the output terminal Q, when the low-level signal is input to the set terminal S from the AND circuit 41. Accordingly, the SRFF 42 continues to output the high-level signal through the output terminal Q, even if the signal which is input to the set terminal S flips from high to low.

The signal which is output through the output terminal Q is input to a gate terminal of the FET 44. Furthermore, a source terminal of the FET 44 is connected to the battery 22 and a drain terminal of the FET 44 is connected to power supply terminals of the devices including the CPU 21, the RAM 24, the light-source device 25, the imaging device 26, and the transmitting circuit unit 27 (hereinafter these devices are referred to as “electronic devices”). The FET 44 is a switching device for powering on or off the electronic devices.

While the FET 44 is switched on by input of the high-level signal from the output terminal g, the electrical power is supplied to the electronic devices from the battery 22 and the electronic devices are driven and operate. On the other hand, while the FET 44 is switched off by input of the low-level signal from the output terminal Q, electrical power is not supplied to the electronic devices and the electronic devices are not driven and do not operate. Namely, the capsule endoscope 10 is turned on by switching the FET 44 on, and turned off by switching the FET 44 off. Alternatively, a relay switch may be utilized instead of the FET 44.

In the initial state, namely before the operation of the CPU 21 starts and before the endoscope 10 is turned on, the low-level signal is output to the SRFF 44 through the output terminal Q so the FET 44 is maintained in the off state and the electronic devices do not operate.

Electric power is always supplied to the logic circuit unit 28 and the UV-EPROM 23 from the battery 22 even in the initial state. Therefore, the logic circuit unit 28 can monitor the signals in the address line A7 and the data line D0 in the initial state.

Each of the address lines A0-A7 connects to the battery 22 through a pull-up resistor 43. In the initial state, signals are not input to the address lines A0-A7 from the CPU 21, but all the address signals in the address lines A0-A7 input to the UV-EPROM 23 are pulled up to the high level by the pull-up resistor 43. Due to this, in the initial state, the binary data of the least-significant bit (Hereinafter referred to “LSB”) of the final byte (0ffh) of the exposable memory field 35B is output to the data line D0 as a data signal from the UV-EPROM 23. Namely, the memory cell 33 corresponding to the LSB in the final byte (0ffh) is addressed from among the plurality of memory cells 35 by the address signals and the binary data of the addressed memory cell 33 is output to the logic circuit unit 2B (the AND circuit 41) as a data signal, through the data line D0.

In the initial state, the electrical charge is stored in the memory cell 33 corresponding to the LSB in the final byte, and therefore, the binary data of the LSB in the final byte is zero. The binary data of the other memory cells 33 in the final byte is random. Accordingly, the final byte is expressed as “*******0b” in binary notation, and “*” means “irrelevant”.

In the initial state, the capsule endoscope 10 is stored in a package (not shown) which is formed of an opaque material such as an opaque film. Due to this, the memory cells 33 in the exposable memory field 35B (namely, final byte 0ffh) do not receive ultraviolet light from the exterior of the shell 11, until the endoscope 10 is removed from the package. Accordingly, the data of the LSB in the final byte (0ffh) is maintained at zero, so the low-level signal is input to the data line D0 and the low-level signal is output from the and circuit 41 to the SRFF 42. while the low-level signal is input from the AND circuit 41 to the SRFF 42, the SRFF 42 continues to output a signal to the FET 44 at low level. Therefore, the operation of the electronic devices is not performed in the initial state.

When the capsule endoscope 10 is used, the endoscope 10 is removed from the package. Then, the ultraviolet light L (shown in FIG. 2) is shone onto the endoscope 10 from the exterior of the shell 11 by the UV-emitting device, and the exposable memory field 35B receives the ultraviolet light. When the memory cell 33 corresponding to the LSB in the final byte (0ffh) in the exposable memory field 35B receives more than a specific amount of ultraviolet light, the value of the LSB in the final byte (0ffh) changes from zero to one. Due to the change in the binary data, the data signal input to the data line D0 flips from low to high. Also, the high-level signal is input to the address line A7 in the initial state, as described above. Therefore, the AND circuit 41 outputs the high-level signal to the SRFF 42.

By inputting the high-level signal, the SRFF 42 outputs the high-level signal through the output terminal Q to the FET 44. The FET 44 is switched on upon receiving the high-level signal. The operation of the CPU 21 starts and then other electronic devices start to be driven and operate also as described above, namely the capsule endoscope 10 is turned on by the switching on of the FET 44, as described above.

After the operation of the CPU 21 starts, the levels of the signals input to the data line D0 and the address line A7 are varied randomly between high and low by the CPU 21. However, after the high-level signal is input to the SRFF 42 from the AND circuit 41 once, the SRFF 42 continues to output the high-level signal even if the signal input to the SRFF 42 flips back to the low level. Therefore, after the operation of the CPU 21 starts, even if the AND circuit 41 outputs the low-level signal with a low-level signal in the address line A7 or the data line D0, the FET 44 continues in the ON state.

That is to say, the logic circuit unit 28 monitors the data signal in the data line D0, and outputs the high-level signal to the PET 44 so as to start the operation of the electronic devices when the monitored data signal flips from low to high. After the operation of the electronic devices starts, the logic circuit unit 28 continues to output the high-level signal to the PET 44 even if the low-level signal is input to the logic circuit unit 28 from the data line D0 or the address line A7. Namely, after the operation of the electronic devices starts, the logic circuit unit 29 does not stop the operation of the electronic devices.

As described above, in this embodiment, the capsule endoscope 10 is turned on by illuminating a part of the UV-EPROM 23 with ultraviolet light from the exterior of the shell 11. The UV-EPROM 23 is basically used for storing the program data. Therefore, the capsule endoscope 10 can be turned on without the special member which is used exclusively for turning on the capsule endoscope 10 and without disassembling the endoscope 10.

Furthermore, when ultraviolet light is used to sterilize the endoscope 10, the endoscope 10 will be turned on Simultaneously with sterilization of the endoscope 10 by illuminating the endoscope 10 with ultraviolet light.

In this embodiment, one byte memory field is allocated to the exposable memory field 35B, but the size of the exposable memory field 35B is not limited. The exposable memory field 35B may be composed of one or more bits (namely, one or more memory calls). Similarly, the size of shielded memory field 35A is not limited, and the shielded memory field 35A may be composed of one or more bits (namely, one or more memory cells).

Furthermore, data such as the program data may be not stored in the shielded memory cells adjoining the exposable memory field 35B, particularly in case of a small exposable memory field 35B, because it is difficult to expose only a few bits of the exposable memory field 35B. In addition, each memory cell 33 may undergo a change in binary data upon receiving more than a specific amount of light of another wavelength instead of ultraviolet light.

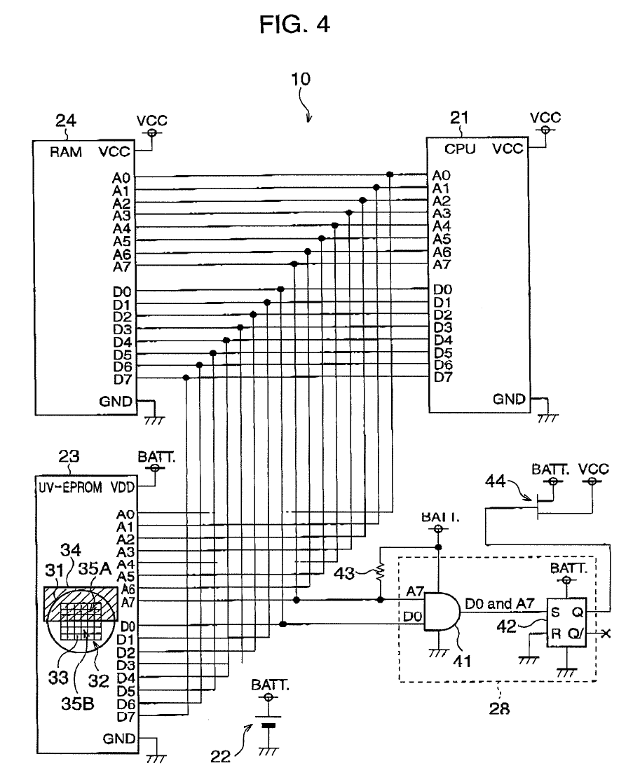

FIG. 4 is a circuit diagram partly showing the capsule endoscope 10 in the second embodiment. The differences between the first and second embodiments will be explained below. In the first embodiment the signals in all of the address lines A0-A7 are pulled up by the pull-up resistor 43, but in the second embodiment the signal only in the address line A7 is pulled up by the pull-up resistor 43.

In the UV-EPROM 23, the first half of the memory field (addresses 00h-7fh), which is a 128-byte memory field, is allocated to the shielded memory field 35A in which the program data is stored, and the second half of the memory field (addresses 00h-0ffh), which is also a 128-byte memory field is allocated to the exposable memory field 35B. In the initial state, the binary value of the LSBs in all of the bytes of the exposable memory field 35B is zero, and all other bits in the exposable memory field 35B are random. Accordingly, all bytes in the exposable memory field 35B are expressed as “*******0b” in binary notation.

In the initial state, the signal in the address line A7 is pulled up to the high level by the pull-up resistor 43, but the signal levels in the other address lines A0-A6 vary randomly between high and low. Therefore, the binary data of the LSB in one byte in the second half field memory (namely, the exposable memory field 35B) is output to the data line D0 from the UV-EPROM 23 as the data signal, but which byte's data is output is not predetermined. Namely, in this embodiment, one memory cell 33 corresponding to the LSB is addressed as the addressed memory cell, but which byte the addressed memory cell is located at is not predetermined.

In the initial state, the exposable memory field 35B does not receive ultraviolet light, as in the first embodiment, and the data of the LSBs of all bytes (80h-0ffh) in the exposable memory field 35B is maintained at zero. Therefore, the data signal input to the data line D0 is maintained at the low level. The AND circuit 41 outputs the low-level signal also. As a result, the operations of the electronic devices are not performed in the initial state.

When the endoscope 10 is used, ultraviolet light is shone on the memory cells 33 in the exposable memory field 35B, as in the first embodiment. When the memory cells 33 corresponding to the LSBs of all bytes in the exposable memory field 35B receive more than a specific amount of ultraviolet light, the data of the LSBs of all bytes in the exposable memory field 35B changes from zero to one. Therefore, the data signal input to the data line D0 is flipped from low to high, although which byte's data is output to the data line D0 is not predetermined.

The FET 44 is switched on when the signal input to the data line D0 is flipped from low to high, thereby starting the operation of the electronic devices, as in the first embodiment. Because the logic circuit 28 has the same structure as that in the first embodiment, the operations of the electronic devices are not stopped by flipping the signal in the address line A7 and data line D0 after the operation of the CPU 21 has begun.

In this embodiment, because the pull-up resistors which are connected to the address lines A0-A6 can be omitted, the circuit structure can be simpler than that in the first embodiment. However, the amount of program data stored in the UV-EPROM 23 is not increased beyond 128 bytes. This embodiment may be useful when the amount of program data is small.

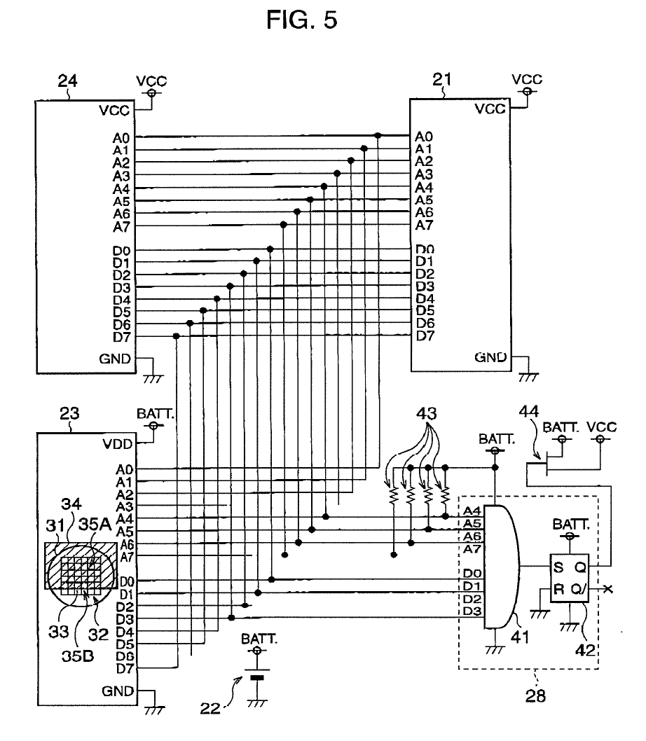

FIG. 5 is a circuit diagram partly showing the capsule endoscope 10 in the third embodiment. The differences between the third embodiment and both the first and second embodiments are explained next.

In the third embodiment, the AND circuit 41 has eight input terminals which connect to the address lines A4-A7 and the data lines D0-D3, respectively. Each address line A4-A7 connects to the battery 22 through the pull-up resistor 43, but the address lines A0-A3 do not.

In the UV-EPROM 23, a 240-byte memory field in the address range 00h-0ffh is allocated to the shielded memory field 35A for storing the program data. A 16-byte memory field in the address range 0f0h-0ffh is allocated to the exposable memory field 35B for altering the operation of the endoscope 10.

In the initial state, binary data of low-order four bits in all bytes of the exposable memory field 35B is zero, because electrical charges are stored in the memory cells 33 corresponding to those low-order four bits. On the other hand, binary data of bits other than those low-order four bits are random. Therefore, all of the bytes (at addresses 0f0h-0ffh) in the exposable memory field 35B are expressed as “****0000b” in binary notation.

In the initial state, the address signals in the address lines A4-A7 are pulled up to the high level by the pull-up resistor 43, but the levels of the address signals in the address lines A0-A3 are randomly between high and low. Accordingly, the data of a specific byte of the exposable memory field 35B (0f0h-0ffh) are output to the data lines D0-D3, but which byte's data among those is not predetermined. However, the data of the low-order four bits in the specific byte are output to the data lines D0-D3 simultaneously. Namely, four memory cells 33 corresponding to low-order four bits at one byte are the addressed memory cells, and their binary data are output to the data lines D0-D3, but which byte the addressed memory cells 33 are located at in the exposable memory field 35B is not predetermined.

In the initial state, the exposable memory field 35B does not receive ultraviolet light, as in the first embodiment, and the data of the low-order four bits of all bytes (0f0h-0ffh) in the exposable memory field 35B is maintained at zero. Therefore, the data signals input to the data lines D0-D3 are maintained at the low level. The AND circuit 41 outputs the low-level signals in the data lines D0-D3 also. Hence, the operation of the electronic devices is not performed in the initial state.

When the endoscope 10 is used, ultraviolet light is shone onto the exposable memory field 35B, as in the first embodiment. When all of the memory cells 33 corresponding to the low-order four bits of all bytes in the exposable memory field 35B receive ultraviolet light exceeding a specific amount, the binary data of the low-order four bits or all bytes in the exposable memory field 35B changes from zero to one. Therefore, all data signals input to the data lines D0-D3 are flipped from low to high, although which byte's data in the exposable memory field 35B is output to the data lines D0-D3 is not predetermined. All address signals in the address lines A4-A7 are at the high level in the initial data, as described above, and therefore, the high-level signals are input to all of the input terminals of the AND circuit 41. Hence, the AND circuit 41 outputs the high-level signal to the SRFF 42. Due to this, The FET 44 is switched on and the operation of the electronic devices starts as in the first and second embodiments.

Furthermore, in this embodiment, the operations of the electronic devices are not stopped by the signal flipping in the address lines A4-A7 and the data lines D0-3 after the operation of the CPU 21 has begun, as in the first and second embodiments.

In this embodiment, the binary data of the four memory cells 33 is monitored by the AND circuit 41, and only when all binary data of the four monitored memory cells 33 changes from zero to one, the operations of the electronic devices start. Therefore, the start of the operation by improper signals is prevented. However, because it is necessary to monitor a lot of data lines, the electrical power consumption in the initial state may increase. Furthermore, because of the several pull-up resistors, the sine of the memory field for storing the program data can be expanded, compared with that in the second embodiment.

Furthermore, the address line(s) may be connected to the battery through the pull-down resistor instead of through the pull-up resistor and the signals in the address line(s) may be pulled down to the low level so as to address one or more than one memory cells 33, in the first to third embodiments.

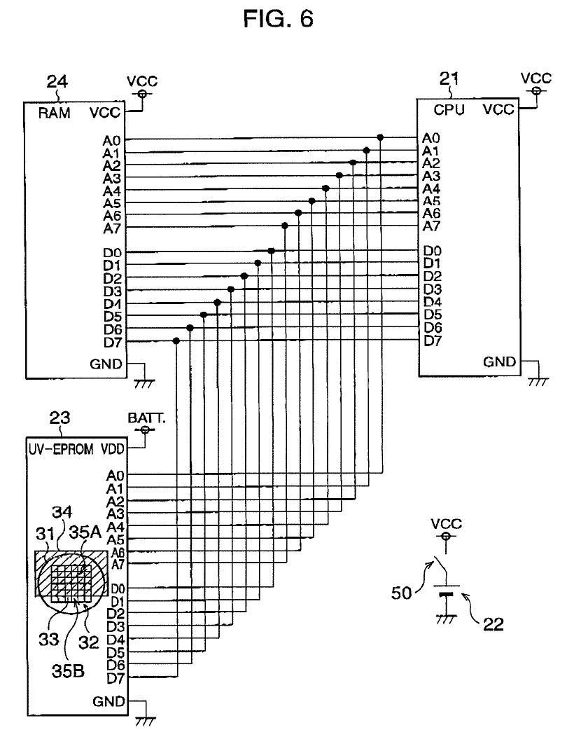

FIG. 6 is a partial circuit diagram for the capsule endoscope 10 of the fourth embodiment. The differences between the third and fourth embodiments are explained next.

In the third embodiment, the exposable memory field 35B is used for turning on the capsule endoscope 10, but in the fourth embodiment the exposable memory 35B is used for another purpose after turning on the capsule endoscope 10. In this embodiment, the amount of ultraviolet exposure in the exposable memory field 35B is detected and the operation of a device in the capsule endoscope 10 is altered according to the detected amount, as described next.

The capsule endoscope 10 has a switch 50 such as a push button. In this embodiment, when the switch 50 is switched on by pushing the button, the electrical power is supplied to the electronic devices from the battery 22. Namely, in this embodiment, the capsule endoscope 10 is turned on by the switch 50 instead of by ultraviolet light.

The UV-EPROM 23 comprises the shielded memory field 35A (addresses 00h-0efh) in which the program data is stored and the exposable memory field 35B (addresses 0f0h-0ffh), as in the third embodiment. In the exposable memory field 35B, the floating gates of all memory cells 33 store electrical charge. Therefore, the binary value of the bits corresponding to all memory cells 33 in the exposable memory field 35B is zero, in the initial state. The binary data of each memory cell 33 changes from zero to one upon receiving more than a specific amount of ultraviolet light, as in the first to third embodiments.

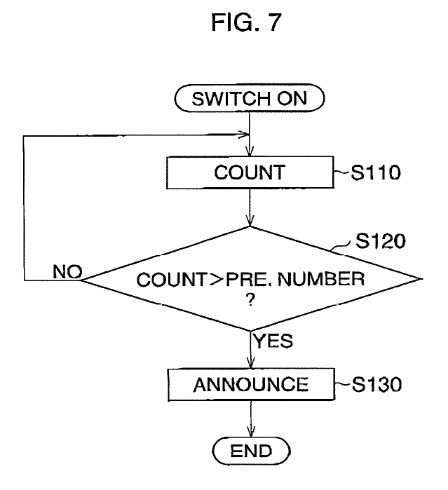

Next, the routine for detecting the amount of ultraviolet exposure will be explained using the flowchart of FIG. 7. This routine starts when the operation of the CPU 21 starts by setting switch 50 to on. Furthermore, when the operation of the CPU 21 starts, the operations of the other electronic devices start, as do those in the first to third embodiments, except that the operation of the light-source device 25 does not start. Therefore, the light-source device 25 does not emit light.

In this routine, at first, the binary data of all bits in the exposable memory field 35B are detected, and then the number of the bits equal to one is counted at step s110. In the initial state, namely before this routine starts, the value of all bits in the exposable memory field 35B is zero. Therefore, the number of the exposable cells whose values have been changed from zero to one by ultraviolet light from the exterior of the shell 11 is counted at step S110.

Next, at step S120, the count is compared with a predetermined number stored in the endoscope 10 in advance. If the count is less than or equal to the predetermined number, the routine comes back to step S110 and the number is counted again. If the count exceeds the predetermined number, it is determined that the capsule endoscope 10 has been exposed by ultraviolet light exceeding a predetermined amount and the routine goes to step S130. At step S130, the light-source device 25 starts to emit light in a specific pattern such as blinking, in order to announce to the user that the ultraviolet exposure has exceeded the predetermined amount and then the routine ends. However, the capsule endoscope 10 may make the announcement in another manner.

As described above, the amount of ultraviolet exposure of the capsule endoscope 10 can be detected by the UV-EPROM 23. Accordingly, for example when the capsule endoscope 10 is sterilized by ultraviolet, the user can judge whether the endoscope 10 has been properly sterilized.

Furthermore, the detection of the amount of ultraviolet exposure may be performed for another purpose. For example, when the count exceeds a specific value, the shutter speed of the imaging device 26 may be altered. Namely, the user may alter the operations of one or more of the electronic devices by illuminating the endoscope with ultraviolet light in a controlled way, after the endoscope 10 has been turned on.

Although the embodiments of the present invention have been described herein with reference to the accompanying drawings, obviously many modifications and changes can be made by those skilled in this art without departing from the scope of the invention.

The present disclosure relates to subject matter contained in Japanese Patent Application No. 2007-289898 (filed on Nov. 7, 2007) which is expressly incorporated herein, by reference, in its entirety.

1. A switching mechanism for altering the operation of an electronic device, said switching mechanism comprising:
a memory comprising a plurality of memory cells, each memory cell undergoing a change in binary data upon receiving more than a specific amount of light of a specific wavelength, the operation being altered when the binary data of at least one memory cell of said plurality of memory cells is changed by said light.

2. The switching mechanism as claimed in claim 1, wherein some memory cell(s) of said plurality of memory cells are shielded such that the shielded memory cell (s) cannot receive said light while the other memory cell(s) are exposable by said light, the operation being altered as the binary data of at least one memory cell of the exposable memory cell(s) is changed by said light.

3. The switching mechanism as claimed in claim 2, wherein specific data is stored in said shielded memory cell(s).

4. The switching mechanism as claimed in claim 3, wherein said specific data constitutes a program, and said electronic device operates based on said program.

5. The switching mechanism as claimed in claim 2, wherein the operation is altered as the number of exposable memory cells whose binary data has been changed exceeds a predetermined number.

6. The switching mechanism as claimed in claim 1, wherein a memory cell is addressed from among said plurality of memory cells, the operation being altered when the binary data of the addressed memory cell is changed by said light.

7. The switching mechanism as claimed in claim 1, wherein two or more memory cells are addressed from among a plurality of memory cells, the operation being altered when the binary data of all of the addressed memory cells is changed by said light.

8. The switching mechanism as claimed in claim 1, wherein said electronic device is a CPU that connects to said memory through a data line and an address line,
said at least one memory cell being addressed by an address signal input to said memory through said address line,
binary data of the addressed memory cell being output as a data signal through said data line, the level of said data signal flipping according to the change in the binary data,
the operation of said CPU being altered when the level of said data signal flips.

9. The switching mechanism as claimed in claim 1, wherein said electronic device is a CPU that connects to said memory through a plurality of data lines and a plurality of address lines,
before the starting operation of said CPU, an address signal in at least one of said address signal lines which is input to said memory being pulled up or down such that binary data of said at least one memory cell is input as a data signal to at least one specific data line of said data lines, the level of said data signal flipping according to the change in the binary data of said at least one memory cell,
the operation of said CPU starting when the level of said data signal flips.

10. The switching mechanism as claimed in claim 1, wherein said switching mechanism further comprises a logic circuit unit,
binary data of said at least one memory cell being input to said logic circuit unit from said memory as a data signal, said data signal flipping from a first level to a second level according to the change in the binary data of said at least one memory cell,
said logic circuit unit altering the operation as said data signal flips from said first level to said second level,
following the altering of the operation, the altered operation continuing to perform even if the level of said data signal flips back to said first level.

11. The switching mechanism as claimed in claim 1, wherein said switching mechanism further comprises a logic circuit unit,
binary data of each of two or more memory cells of said plurality of memory cells being input to said logic circuit unit from said memory as a data signal, the level of said data signal flipping from a first level to a second level according to the change in the binary data of each of said two or more memory cells,
said logic circuit unit altering the operation when all of said data signals are at said second level.

12. The switching mechanism as claimed in claim 1, wherein said switching mechanism is provided, in a swallowable medical device.

13. The switching mechanism as claimed in claim 12, wherein said swallowable medical device is a capsule endoscope.

14. The switching mechanism as claimed in claim 1, wherein said electronic device and said memory are provided in the interior of a shell, said shell comprising a transparent portion that transmits light of said specific wavelength from the exterior of said shell to the interior of said shell,
said at least one memory cell receiving said light passing through said transparent portion from the exterior of said shell.

15. The switching mechanism as claimed in claim 14, wherein an objective lens system is provided in said interior, said objective lens system isolated from light directed from outside said shell to said memory.

16. The switching mechanism as claimed in claim 1, wherein said memory its a UV-EPROM.
